(19) United States
(12) Patent Application Publication
(10) Pub. No.: US 2004/0043063 A1
(43) Pub. Date: Mar. 4, 2004

(54) Expression of keratin genes 8 and 18 for treating tumours,in particular a mammary carcinoma

(76) Inventor: Gerhard Schaller, Berlin (DE)

Correspondence Address: BIRCH STEWART KOLASCH & BIRCH, PO BOX 747, FALLS CHURCH, VA, 22040-0747, US

(21) Appl. No.: 10/433,772

(22) PCT Filed: Dec. 7, 2001
(86) PCT No.: PCT/DE01/04579

(30) Foreign Application Priority Data
Dec. 7, 2000 (DE) 100 61 110.9

(51) Int. Cl. A61K 48/00; A61K 39/395; A61K 9/127

(52) U.S. Cl. 424/450; 424/143.1; 514/44

(57) ABSTRACT

The invention is concerned with a means for the therapy of malignant diseases, in particular for the prevention or inhibition of metastases of solid tumours expressing HER2. The means in keeping with this invention is characterized by the fact that it favours the expression of the Keratin genes 8 and 18 (K8, K18) in tumour cells. The K18 gene is preferred for the gene therapeutic treatment and it is preferential for the HER2-mediated in vivo transfection with a liposomal capsule equipped with antibodies against the HER2 receptor. The subject of the invention is thus the use of the K8 and K18 genes for the therapy of solid tumours, in particular for breast cancer. Areas of application for the invention are in medicine and the pharmaceutical industry.

### DESCRIPTION

The invention concerns a means of treatment of malignant disease and especially for the prevention or inhibition of metastasis in HER2 expressing solid tumours. The means invented is characterized by the fact that it favours the expression of keratin 8 and 18 (K8, K18) genes in tumour cells. It is primarily concerned with the use of the K18 gene in gene therapy. This is available bound to a liposome capsule possessing antibodies to the HER2 receptor, for the purpose of HER2 mediated in vivo-transfection. What has been invented is thus the use of the K8 and K18 genes for the treatment of solid tumours, primarily breast cancer. Areas of application for the invention are medicine and the pharmaceutical industry.

Breast cancer is the most common malignant disease in women. The fate of patients is decided by the process of metastasis. A first step in the development of metastases is the impairment of tissue integrity and an increase in cellular plasticity. The immediate substrate of these functional changes in the tumour cells are the proteins of the cellular adhesion structures and the cytoskeleton. Among the many structures that have been described so far, an outstanding role appears to be played by the e-cadherin complex of the adherens junctions, the functional proteins of the desmosomes and the keratins of the intermediat filaments anchored here.

The keratins here are a group consisting up to now of 20 known proteins. Expression is both tissue- and function-specific (Franke 1981). In the epithelial cells the keratins are usually expressed pairwise, as heterodimers making up the intermediat filaments which are themselves a part of the cytoskeleton. The keratin pair 8 and 18 (K8/18) is thus always present in the luminal cells of the two-layer mammary gland epithelium, representing specific differentiation. This strict pairwise expression has meant that, for methodological reasons, scientific investigations have concentrated on either one keratin or the other (Baribault 1993). From the behaviour of one keratin, conclusions can always be drawn regarding the behaviour of its partner. It is not possible for a functioning filament to be formed exclusively from K8 or K18.

In the formation and development of solid tumours in particular, suppression of keratins associated with differentiation usually occurs in the course of malignant transformation. This loss is balanced by an increased expression of other keratins and of vimentin. The substitution of the keratins 8/18 with the mesenchymal intermediat filament protein vimentin is associated with a loss of tissue integrity, increased cell plasticity and, as a result, an increasing tendency for metastasis, as has been shown by Vallés and Thiery for bladder carcinoma (Vallés 1990). Experiments by Oshima's team have demonstrated the importance of the expression of K8/18 for tissue integrity: a knockout mutation of the K8 gene in mice led to the intrauterine death of embryos as a result of bleading from inadequately structured liver tissue (Baribault 1993).

From these investigations it follows that the loss of K8/18 is associated with a reduction in tissue integrity and also with increased plasticity in the affected cells. These changes in cell properties also explain the high rate of metastasis in tumours whose cells demonstrate reduced K8/18 expression. For breast cancer it was likewise possible to demonstrate that the expression of vimentin in the tumour cell is associated with aggressive metastasis and thus leads to a poor prognosis (Thompson 1992, Sommers 1994).

The tendency to metastasis is accepted as a sign of malignancy in the classification of tumours and is one reason why many cancer disorders are still incurable today in spite of the enormous progress of science and research in this area in recent years. The established treatment methods used up to now, which concentrate on surgical removal of tumour tissue and suppression of cell division by chemotherapy or radiation, appear to be unable to produce a significant improvement in the chances of healing malignant cancer disorders.

Gene therapy promises better results. This relatively new therapeutic principle targets the source of the problem and attempts to repair the defects in genes, or pathogenic regulatory mechanisms, that cause tumours to form. The complexity of the formation of most tumours makes it difficult to carry out targeted repairs on defective genes. In addition, ensuring the effective insertion of the genes into the cells to be treated continues to present difficulties.

Receptors that are present in increased numbers on the surface of the tumour cells, thus identifying the cells clearly, promise great progress in the improvement of insertion of the genes relevant to therapy. HER2 (Human Epidermal Growth Factor Receptor) is over-expressed in 25% of all breast cancers. During embryogenesis HER2 is involved in the development of the heart and brain. It is only residually expressed in the adult organism. Patients with this tumour marker have a particularly bad prognosis because this receptor, which belongs to the EGFR family, transmits strong proliferation signals to the cell nucleus on the one hand and on the other, by destruction of cell-cell contacts in the epithelia, leads to particularly aggressive metastasis.

The therapeutic goal of the established therapies, such as operation, chemotherapy, radiotherapy and hormone therapy, and also of inhibitors of neoangiogenesis and tyrosine kinase, currently undergoing preclinical trials, and the already licensed Trastuzumab (Herceptin™), is the inhibition of proliferation.

This disregards the fact that, in many cancer disorders, it is above all the results of the loss of differentiation in the form of the growth of metastases which is responsible for the frequent occurrence of relapse and thus for the failure of an effective therapy.

A fundamental disadvantage of the therapeutic methods aimed at inhibiting proliferation is, therefore, that these methods are not able to ensure prevention or effective suppression of tumour metastasis.

For these reasons the basic task for the invention was to find and make available for use a means for the treatment of solid tumours using gene therapy. As well as inhibiting proliferation this means was required to give effective suppression of metastasis formation either by inducing differentiation or by preventing loss of differentiation.

In a retrospective study of 134 ductal breast cancers, an immunohistochemical analysis showed that K8/18 expression in the tumour is correlated with patient survival (Leube 1986, Yamamoto 1990). About 1 patient in 5 was positive (score>4 out of a maximum of 12). All except one of these patients were free of metastases after an observation period of 9 years. A similar correlation was found in a parallel study of (up to now) 70 patients with endometrial carcinoma (Schaller 1996).

These findings were also supported in vitro. Using several established breast cancer cell lines, investigations were carried out of K18 expression, invasivness in the Boyden chamber and metastasis in the athymic nude mouse. Here, too, a close inverse correlation was shown between K18 expression and malignity.

The invention is based on our own findings that K18 expression in each tumour that develops from a two-layer epithelium represents a clear obstacle to invasion and metastasis. In cultivated breast cancer cells an increased K18 expression is correlated with dramatically reduced malignity and cell aggressivity. This makes clear once again that a high level of K18 expression in a tumour is associated with a significantly better prognosis, for example for breast cancer patients.

The major requirements specified for the invention are achieved while the secondary requirements represent variations according to preference. What has been invented is primarily a means for the treatment of tumours using gene therapy which either causes over-expression of the K8 or K18 gene in the tumour, or which itself proves the K8 or K18 gene. This is introduced into the tumour using known gene therapy methods and there brings about overexpression. The combination of both genes also represents an alternative form. For gene therapy the genetic substance is available for direct application in formulations for parenteral use, which are favoured, or in the form of liposomal complexes, which are especially favoured. In another variant the nucleotide is made available for use in gene therapy in the form of a vector. In a further variant the means invented contains the K8 and/or K18 protein. With this invention it has been possible for the first time to show that the K8 and K18 genes and their proteins are associated with differentiation and are therefore diametrically opposed to malign transformation.

In one preferred version of the invention, the invented means, K8 or K18 gene is inserted into an already known liposome capsule possessing HER2 antibodies as a carrier molecule. With this carrier it can be introduced into the tumour cell where it brings about overexpression. The liposome capsule has a strong cationic charge and has antibodies to the HER2 receptor bound to it. Its use for the introduction of chemotherapeutic agents and genetic constructions into tumour cells has already been described. Parts of an antibody, e.g. Fab fragments of Trastuzumab (Herceptin™), a humanized antibody produced using genetic technology and licensed as a drug in Europe since 28.08.2000, are also suitable for the recognition of tumour cells. After the HER2-liposome combination has bound to the antigen it is internalized by the tumour cell and the contents of the liposome capsule are released (Kirpotin 1997, Park 1997). Up to now, however, its application has been limited to inhibition of proliferation.

Gene therapy as invented here with the K8 or K18 gene, especially with the K18 gene, surprisingly makes possible a combination of reliable inhibition of proliferation with induction of differentiation. In this way a reliable and lasting treatment is possible for all solid tumours that develop from a two-layer epithelium, and above all for breast cancer. This treatment is not limited to a HER2 mediation of the genes.

It is thus possible, according to the invention, to intervene in the tumour cell during the GO phase of the cell cycle and to induce differentiation or to prevent a loss of differentiation, making it possible to effectively suppress the formation of metastases. Depending on the degree of HER2 expression in the tumour and strong bystander effects, a drastic (>90%) reduction of malignity can be achieved.

The advantages of the invented means for gene therapy using HER2 mediated K8/K18 transfection are:

1. In contrast to proliferation suppression, tumour cells in the GO phase are also accessible to HER2 mediated gene therapy.

2. This therapy is, in principle, applicable to all solid tumours expressing K8 and/or K18 in the epithelium of origin and HER2 in the tumour. This is the case for all tumours that develop from a two-layer epithelium.

3. The induction of differentiation is in accordance with physiological development and compensates for the malign loss of differentiation.

### LITERATURE:

Baribault H. et al., Mid-gestational lethality in mice lacking keratin 8 Genes & Development 7 (1993) 1191

Kirpotin D. et al., Sterically stabilized anti-HER2 immunoliposomes: Design and targeting to human breast cancer cells in vitro Biochemistry 36 (1997) 66

Leube R E. et al., Cytokeratin expression in simle epithelia III. Detection of mRNAs encoding human cytokeratins nos. 8 and 18 in normal and tumor cells by hybridisation with cDNA sequences in vitro and in situ Differentiation 33 (1986) 69

Moll R. et al., The Catalog of human cytokeratins: patterns of expression in normal epithelia, tumors and cultured cells Cell 31 (1982) 11

Nakamura Y., Cleaning up on β-catenin Nature medicine 3 (1997) 499

Park J. W. et al., Anti-HER2 immunoliposomes for targeted therapy of human tumors Cancer Letters 118 (1997) 153

Thompsom E. W. et al., Association of increased basement membrane invasiveness with absence of estrogen receptor and expression of vimentin in human breast cancer cell lines J Cellular Physiology 150 (1992) 534

Schaller G. et al., Elevated Keratin 18 protein expression indicates a favorable prognosis in patients with breast cancer Clin Cancer Res 2 (1996) 1879

Slamon D. J. et al., Human Breast Cancer: Correlation of Relapse and Survival with Amplification of the HER-2/neu Oncogene Science 235 (1987) 177

Vallés A. M. et al., Acidic fibroblast growth factor is a modulator of epithelial plasticity in rat bladder carcinoma cell line Proc Natl Acad Sci USA 87 (1990) 1124

Yamamoto R. et al. Cloning and Swquence of cDNA for Human Placental Cytokeratin 8. Regulation of the mRNA in Trophoblastic Cells by cAMP Molecular Endocrinology 4 (1990) 370

In the following, the invention is explained in more detail using the model examples for K18.

### MODEL EXAMPLES

### Example 1

By transfecting the K18 gene into the breast cancer cell line MDA MT 231 an increase of the K18 expression was obtained, whereby a reduced malignancy of the tumour cells was achieved, which is characterized by:

a) an increased cellular adhesiveness,

b) induced adhesion proteins,

c) lower proliferation in the soft agar,

d) lower invasiveness in the Boyden chamber, and

e) smaller tumours in the nude mouse.

### Example 2

Further investigations with a K18 positive sub-clone, which is normally K18 negative but however now transfected with the K18 gene, resulted in a drastically (>90%) reduced malignancy of the K18 positive clone in the form of an increased expression of desmosomal proteins, a lack of invasiveness in the Boyden chamber and a lack of metastases in the nude mouse.

1. Agent for the therapeutic treatment of tumours by simultaneously inhibiting and preventing tumour metastases, characterised by the fact that it increases the expression of the K8 and/or K18 gene in the tumour cells.

2. Agent according to claim 1, characterized by the fact that it represents the K8 and/or K18 gene or therapeutically effective sections of it, which are transfected in the tumour cells, preferably in the G0 phase of the cell cycle.

3. Agent according to claim 1 or 2, characterized by the fact that it is suitable for transfection in tumour cells, which display the HER2 receptor on the cell surface.

4. Agent according to one of the claims 1 to 3, characterized by the fact that the genetic substance is present for direct application, preferably in formulations for parenteral application or in the form of liposomal complexes.

5. Agent according to one of the claims 1 to 3, characterized by the fact that the nucleotide is present in the form of a vector for gene therapeutic application.

6. Agent according to one of the claims 1 to 3, characterized by the fact that it is the K8 and/or K18 protein.

7. Agent according to claim 4, characterized by the fact that the liposomal complex is a capsule of liposomes equipped with antibodies against the HER2 receptor.

8. Agent according to claims 4 or 7, characterized by the fact that the liposomal capsule is strongly cationic-loaded.

9. Agent according to claim 8, characterized by the fact that Trastuzumab (Herceptin™) is used as a carrier.

10. The use of agent according to the claims 1 to 9 for the gene therapy of solid tumours, which have developed from simple epithelia.

11. The use according to claim 10 for the gene therapy of solid tumours that express HER2.

12. The use according to claims 10 or 11 for the gene therapy of breast cancer.

13. Procedures for the production of a pharmaceutical agent agent according to one of the claims 1 to 9, characterized by the fact that the therapeutic quantity of at least one nucleotide or protein is combined with currently known auxiliary and carrier substances, manufactured by the current and usual production techniques, and this combination is then transferred into the desired formulation.
